(19) United States
(12) Patent Application Publication
(10) Pub. No.: US 2015/0238533 A1
(43) Pub. Date: Aug. 27, 2015

(54) Composition and Method of Treating Eye Discharge

(71) Applicant: Karen Opp, Salida, CO (US)

(72) Inventor: Karen Opp, Salida, CO (US)

(21) Appl. No.: 14/623,261

(22) Filed: Feb. 16, 2015

Related U.S. Application Data
(60) Provisional application No. 61/942,729, filed on Feb. 21, 2014.

(51) Int. Cl. A61K 35/34

(52) U.S. Cl. CPC A61K 35/34

(57) ABSTRACT

A composition and method of treating eye discharge for an animal is provided. The composition and method includes providing a dehydrated lamb heart and a dehydrated beef heart. The dehydrated lamb heart and the dehydrated beef heart are combined to form a dehydrated lamb and beef heart mixture. Effective amounts of the dehydrated lamb and beef heart mixture are orally administered to an animal. Dehydrated lamb and beef hearts contain essential vitamins and minerals that help treat and prevent eye discharge in animals.

### CROSS REFERENCE TO RELATED APPLICATION

This application claims the benefit of U.S. Provisional Application No. 61/942,729 filed on Feb. 21, 2014. The above identified patent application is herein incorporated by reference in its entirety to provide continuity of disclosure.

### FIELD OF THE INVENTION

The invention generally relates to a composition and method of preventing accumulation of eye discharge for an animal. More specifically, the present invention relates to a method of orally administering a dehydrated beef and lamb heart mixture in order to reduce tearing and tear stains.

### BACKGROUND OF THE INVENTION

The invention relates to management of tear stains (e.g., in dogs). More particularly, the invention relates to nutritional/dietary supplements for assisting in such management. Tear staining has been a significant problem in dogs and cats. It has been a particular problem in certain light-coated breeds where the stains are more visible. Bacterial infection is the most common reason behind increased tearing and resultant stains.

The eye discharge may cause staining around the eyes, as well as swelling around the eyes. The accumulation of eye discharge and associated discomfort experienced by the pet may cause the pet's eyes to appear dull and lackluster and may negatively impact the pet's vision. In order to prevent the accumulation of eye discharge, the pet's owner may have to manually clean or wipe the discharge from the pet's eye by hand, which can be inconvenient. Manually removing the pet's eye discharge temporarily alleviates the problem, as the real cause of the eye discharge may result from a bacterial infection, glaucoma, or allergies.

Some compositions in the prior art have been disclosed that relate to tear stain management compositions and methods, such as U.S. Published Patent Application Number 2013/0195868, U.S. Pat. No. 7,964,225, and U.S. Pat. No. 8,497,094. Other compositions relate to emulsion impregnated rawhide chews provided in an aqueous based soaking operation, such as U.S. Pat. No. 7,147,888.

These prior art compositions and methods have several known drawbacks. Such compositions and methods fail to provide a dehydrated lamb heart and a dehydrated beef heart that are combined to form a dehydrated lamb and beef heart mixture. The prior art fails to provide methods for treating eye discharge that involve oral administration of effective amounts of the dehydrated lamb and beef heart mixture.

In light of the compositions and methods disclosed in the prior art, it is submitted that the present invention substantially diverges from the prior art and consequently it is clear that there is a need in the art for an improvement to existing eye discharge treatment compositions and methods. In this regard the instant invention substantially fulfills these needs.

### SUMMARY OF THE INVENTION

In view of the foregoing disadvantages inherent in the known types of tear staining remedies now present in the prior art, the present invention provides a method for treating eye discharge wherein the same can be utilized for providing convenience for the user when treating a pet with excessive eye discharge and tear staining.

It is therefore an object of the present invention to provide a new and improved method of treating eye discharge for an animal that has all of the advantages of the prior art and none of the disadvantages.

One object of the present invention is to provide a method of treating eye discharge, wherein the method includes the steps of providing a dehydrated lamb heart and a dehydrated beef heart, combining the ingredients into a mixture, and orally administering an effective amount of the mixture to an animal.

It is another object of the present invention is to provide a method for treating eye discharge including providing a dehydrated beef heart and a dehydrated lamb heart.

It is another object of the present invention is to provide a method for treating eye discharge including combining the dehydrated lamb heart and the dehydrated beef heart to form a dehydrated lamb and beef heart mixture. The dehydrated lamb and beef heart mixture may comprise 50 weight percent dehydrated lamb heart and 50 weight percent dehydrated beef heart.

Another aspect of the present invention is to provide a method for treating eye discharge including orally administering an effective amount of the dehydrated lamb and beef heart mixture to an animal.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a flowchart of the method for treating eye discharge according to one embodiment of the present invention.

### DETAILED DESCRIPTION OF THE INVENTION

Reference is made herein to the attached drawings. Like reference numerals are used throughout the drawings to depict like or similar elements of the bracket device of the present invention. For the purposes of presenting a brief and clear description of the present invention, the preferred embodiment will be discussed as used for treating eye discharge and staining for pets. The figures are intended for representative purposes only and should not be considered to be limiting in any respect.

The beef and lamb hearts are high in riboflavin, B-12, selenium, thiamin, phosphorus, zinc, iron, copper, pantothenic acid and niacin. The effective amount of B-12 in the dehydrated beef and lamb heart mixture monitors the level of oxygen throughout the body and, as an antioxidant, it protects white blood cells used by the immune system to kill bacteria in the eye. Enzymes depend on the presence of iron to complete biochemical reactions that make collagen and neurotransmitters. B-12 activates chemical reactions that turn fat into energy and help cells fight carcinogens.

Additionally, the copper in the beef and lamb hearts promotes healing of damaged tissue. Copper stimulates the immune system to fight infections and repair injured tissues. Copper helps to neutralize free radicals which can cause severe damage to cells. The zinc contained in the hearts helps copper to be absorbed more efficiently.

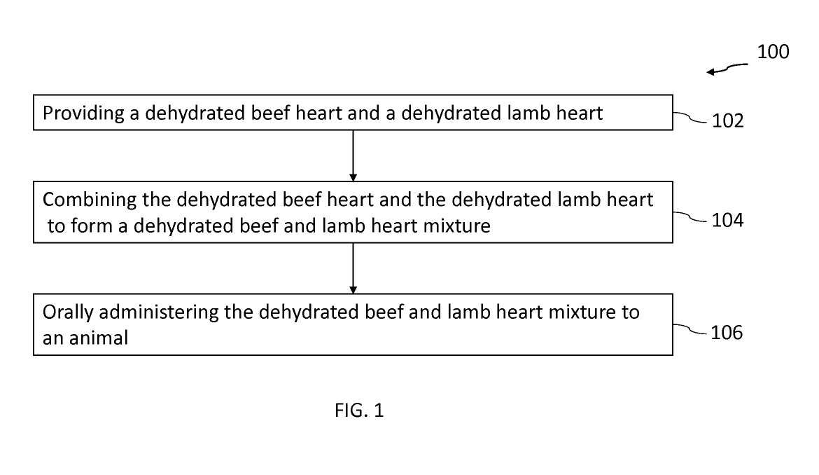

Referring now to FIG. 1 there is shown a flowchart of a method for treating eye discharge according to one embodiment of the present invention. At providing step 102, a dehydrated beef and a dehydrated lamb heart is provided. The beef and lamb hearts are preferably dehydrated by smoking, freeze-drying, or curing with salt, however, in alternate embodiments other means for dehydrating the lamb and beef hearts may be utilized. Once the lamb and beef hearts are dehydrated, the lamb and beef hearts can be stored in vacuum sealed bags or containers. It is also contemplated that natural beef and lamb hearts may be provided without dehydrating the hearts.

After providing step 102, the method continues to combining step 104. At combining step 104, the dehydrated beef and the dehydrated lamb heart is combined to form a dehydrated beef and lamb heart mixture. The beef and lamb hearts can be shredded or pulverized using any conventional food processing means, such as a blender or food processor. The beef and lamb hearts are then provided as grains or small chunks than be can molded or formed into pellets or the like. In a preferred embodiment, the amount of a dehydrated lamb heart is at least 50 weight percent of the total composition. The combination can be provided in a powder form or bite sized chunks.

An exemplary embodiment of the dehydrated beef and lamb heart mixture is 50% of each the dehydrated beef heart and dehydrated lamb heart, by weight. Depending on the severity of the eye discharge, the weight percentage of each component in the mixture is adjusted, or the dosage may be increased. The dosage of the dehydrated beef and lamb heart mixture is also altered according to the ailment being treated, such as conjunctivitis, epiphora, and dry eyes. Exemplary packaging for the dehydrated beef and lamb heart mixture includes plastic or glass jars that provide an air tight closure.

After combining step 104, the method continues to administering step 106. At administering step 106, effective amounts of the dehydrated lamb and beef mixture are administered to an animal. The dehydrated beef and lamb heart mixture can be administered via alternative delivery vehicles such as edible chews, capsules, pills, tablets, and the like. In an alternate embodiment, the dehydrated lamb and beef mixture further includes additives such as proteins, minerals, and flavorings.

The beef and lamb hearts are high in riboflavin, B-12, selenium, thiamin, phosphorus, zinc, iron, copper, pantothenic acid and niacin. The effective amount of B-12 in the dehydrated beef and lamb heart mixture monitors the level of oxygen throughout the body and, as an antioxidant, it protects white blood cells used by the immune system to kill bacteria in the eye. Enzymes depend on the presence of iron to complete biochemical reactions that make collagen and neurotransmitters. B-12 activates chemical reactions that turn fat into energy and help cells fight carcinogens.

One or more embodiments of the present invention have been described. Nevertheless, it will be understood that various modifications may be made without departing from the spirit and scope of the invention. For example, alternative delivery vehicles such as edible chews, capsules/pills/tablets, and the like are possible. Accordingly, other embodiments are within the scope of the following claims.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A method for treating eye discharge in an animal, comprising the steps of:
providing a dehydrated lamb heart and a dehydrated beef heart;
combining the dehydrated lamb heart and the dehydrated beef heart to form a dehydrated lamb and beef mixture;
orally administering effective amounts of the dehydrated lamb and beef mixture to an animal.

2) The method of claim 1, wherein the dehydrated lamb heart is at least 50 weight percent of the total composition.

3) The method of claim 1, wherein dehydrated lamb and beef mixture further includes one or more additives selected from the group consisting of proteins, minerals, and flavorings.

4) The method of claim 1, wherein the step of combining the dehydrated lamb heart and the dehydrated beef heart includes the step of grinding said dehydrated lamb heart and a dehydrated beef heart into a powder.

5) The method of claim 1, wherein the step of combining the dehydrated lamb heart and the dehydrated beef heart includes the step of suspending the dehydrated lamb heart and the dehydrated beef heart in solution.

6) A composition for treating eye discharge in animals, comprising:
an effective amount of dehydrated lamb heart;
an effective amount of dehydrated beef heart.
